(19) United States
(12) Patent Application Publication
(10) Pub. No.: US 2009/0012614 A1
(43) Pub. Date: Jan. 8, 2009

(54) DEVICE AND METHOD FOR TETHERING A SPINAL IMPLANT

(76) Inventor: Robert A. DIXON, Dublin, OH (US)

Correspondence Address: STANDLEY LAW GROUP LLP, 495 METRO PLACE SOUTH, SUITE 210, DUBLIN, OH, 43017, US

(21) Appl. No.: 12/117,358

(22) Filed: May 8, 2008

Related U.S. Application Data
(60) Provisional application No. 60/916,635, filed on May 8, 2007.

(51) Int. Cl. A61F 2/08; A61B 17/08

(52) U.S. Cl. 623/13.14; 606/151

(57) ABSTRACT

A device for tethering an implant device along the posterior spine of a human has first and second elements, as well as a means for fixing, in a quick connect manner, the first and second elements to each other. The first element is adapted for being secured to an interspinous ligament and the second element is adapted for attachment to the implant device. The first element may be a beam that is adapted for placement through the interspinous ligament in a direction substantially horizontal to a long axis of the spine.

### CROSS-REFERENCE TO RELATED APPLICATIONS

This application is a non-provisional application that claims a benefit of priority, under 35 USC 120, to provisional application Ser. No. 60/916,635, filed on 8 May 2007, which is incorporated by reference as if fully recited herein.

### TECHNICAL FIELD

Generally, the disclosed embodiments herein relate to a device and a method for tethering a spinal implant (e.g. tube or wire) to a mammalian, and particularly, a human spine.

### BACKGROUND OF THE ART

Certain terms are defined for their use herein. When discussing the spine, the term “dorsal” or “posterior” refers to the back of the spine. The spinous process points in the dorsal or posterior direction. Depending upon the location in the spine, the spinous process may also be directed somewhat caudally in the longitudinal direction. The term “ventral” or “anterior” refers to the front of the spine, that is, the side opposite the dorsal or posterior side. The term “interspinous” refers to a region between the respective spinous processes. The term “supraspinous” refers to the region dorsal to the spinous processes. “Flexion” of the spinous processes occurs when the spinous processes move away or apart from each other. A result of this is elongation or stretching of the associated ligaments. “Extension” of the spinous processes refers to movement towards each other. When the spinous processes extend, the associated interspinous ligaments delongate or contract. A “lateral” or “side” view is a view from a right towards the left side of a patient. The long axis of the spine, referred to as the “longitudinal axis” or the “spinal axis” runs approximately in the direction from the head to the tailbone, with the direction toward the head referred to as being “cephalad” and the direction toward the tailbone referred to as being “caudad.” The “spinal canal” is the space within the spinal column through which the neural elements (the nerves and spinal cord) traverse. “Horizontal” refers to the direction perpendicular (radial) to the longitudinal axis of the spine. “Right” and “left” are relative terms used to describe a direction perpendicular to the longitudinal axis of the spine.

As the devices described herein are place on the spinous process side of the spine, reference to the “ventral side” of a device is to the side that is placed deepest into the patient, that is, closest to the spine, and the “dorsal side” of the device is the side which is most superficial, that is, furthest from the spine. A “front view” refers to the direction looking along the long axis of the device, from one end to the other. An “enlarged view” does not refer to a particular degree of magnification, and a “focused view” refers to viewing a portion of the device, not the device in whole.

For the purposes of clarification, a “spinal implant” refers to the implant to be attached to the spine by the device and method. The terms “tethering device” or “device” refers to this patent device. An “anchoring implant” refers to a prior art device used to anchor a spinal implant, typically utilizing sutures or ligatures.

Turning now to the unsolved problem of the prior art, an increase in the number of surgical spinal implants using wires and tubes has been associated with improved technologies to maintain these wires and tubes in proper cephalad-caudad (cephalo-caudal) position within or around the spinal canal. Maintaining proper position, during both surgery and during post-operative spinal motion, reduces the risk of spinal implant migration and spinal implant breakage. Either of these undesirable effects can result in failure of the spinal implant function.

In the prior art, the spinal implant is typically sutured to the spinous process or surrounding fascia using an anchoring implant. The anchoring implant is independently sewn to the spinal implant using a ligature type stitch. This method is surgically cumbersome because it requires suturing the anchoring implant to the spinal implant, followed by suturing the anchoring implant to the spine. This adds time to the surgery and requires a larger incision to accommodate the suturing. Suturing can be inconclusive in regards to proper spinal implant tethering. Additionally, suturing produces a focal fixation point which can increase the stress riser on the implant, as opposed to a more diffused attachment means such as a tunnel.

It is therefore, an unachieved advantage of the prior art to provide a device that uses quick connect mechanisms and does not require suturing to attach spinal implants to the spine. In the most preferred embodiments, the device would not only do this, but would also maintain the position of the spinal implant, provide a more assured tethering attachment, provide minimally invasive placement of the spinal implant, and reduce repetitive stress upon the spinal implant.

### SUMMARY OF THE INVENTION

These unmet advantages of the prior art are achieved by the device as described in claim 1.

### BRIEF DESCRIPTION OF THE DRAWINGS

Better understanding will be had of the embodiments of the invention when reference is made to the accompanying drawings, wherein identical parts are identified with identical reference numbers and wherein:

FIG. 1 is a lateral view of a portion of a mammalian spine;

FIG. 2 is a top view of the spinal portion of FIG. 1;

FIGS. 3 to 9 are side elevation views of seven embodiments of a first or horizontal element used in the tethering device;

FIG. 10 is a plan view of the dorsal surface of a spine with a tethering device implanted;

FIG. 11 is a side elevation view of the FIG. 10 spine with implanted tethering device;

FIG. 12 is an end elevation view of the FIG. 10 spine with implanted tethering device, taken along the longitudinal axis of the spine;

FIG. 13 is an end elevation view of a variation of the tethering device of FIG. 11;

FIG. 14 is a plan view of the dorsal surface of the FIG. 12 tethering device with the implant connectors attached;

FIG. 15 is an enlarged lateral view of the tethering device shown in FIG. 13;

FIGS. 16A through 16E illustrate some methods of attaching an implant connector to a first element to form a tethering device;

FIGS. 17A and 17B are front elevation views of a third embodiment of an implant connector, FIG. 17A showing the through holes in an “open” position and FIG. 17B showing the through holes in a “closed” position;

FIGS. 18A and 18B are side elevation views of the FIGS. 17A and 17B implant connector;

FIGS. 19A and 19B are side elevation views of a further combination of a horizontal element and an implant connector, with FIG. 19A showing the implant connector not connected to the horizontal element and with FIG. 19B showing the implant connector connected;

FIG. 20 is a plan view of the dorsal surface with a horizontal element/implant connector combination of FIG. 19 positioned in an interspinous ligament;

FIGS. 21A and 21B are side elevation views of a further combination of a horizontal element and an implant connector, with FIG. 21A showing the implant connector not connected to the horizontal element and with FIG. 21B showing the implant connector connected;

FIG. 22 is a plan view of the dorsal surface with a horizontal element/implant connector combination of FIG. 21 positioned in a spinous process;

FIGS. 23A and 23B are enlarged end elevation views of the implant connector and horizontal element of FIGS. 19A and 19B, with FIG. 23A corresponding to the FIG. 19A view and FIG. 23B corresponding to the FIG. 19B view;

FIGS. 24A and 24B are side sectional elevation views of a further combination of a horizontal element and an implant connector, with FIG. 21A showing the implant connector not connected to the horizontal element and with FIG. 21B showing the implant connector connected;

FIG. 25 is a plan view of the dorsal surface of a spine with another embodiment of a tethering device implanted;

FIG. 26 is a side elevation view of the FIG. 25 spine with implanted tethering device;

FIG. 27 is a front elevation view of a tethering device with the implant connector integral with the horizontal element;

FIGS. 28A and 28B are side elevation views of the FIG. 27 tethering device, with FIG. 28A showing the device penetrated in an open condition in an interspinous ligament and with FIG. 28B showing the device engaged in a closed condition;

FIGS. 29A, 29B and 29C show, in a view taken along the spinal axis, a sequence of engaging the FIG. 27 tethering device into the interspinous ligament using a tool;

FIGS. 30A, 30B and 30C show, in a view taken along the spinal axis, a sequence of engaging a tethering device of FIG. 30D into the interspinous ligament using a tool that is similar to the FIG. 30 tool.

### DETAILED DESCRIPTION OF A PREFERRED EMBODIMENT

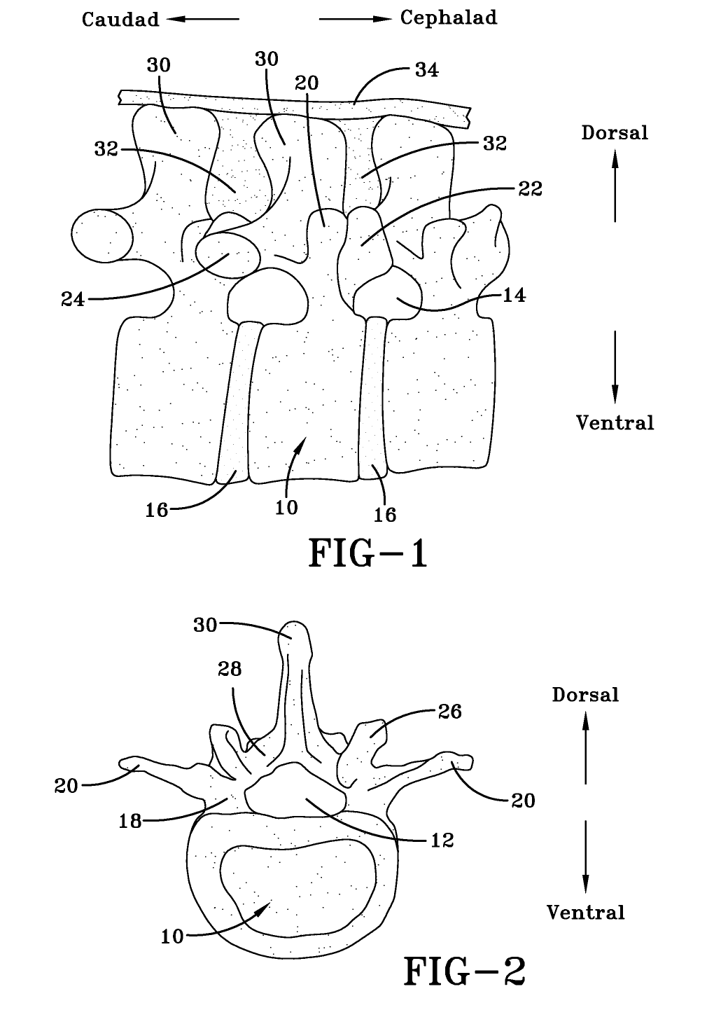

To provide a better understanding of the environment in which the embodiments to be described are used, FIGS. 1 and 2 provide a side and top view of a portion of a mammalian spine, specifically a lumbar spine portion. FIG. 1 shows the side view of a spinal portion of a patient in the prone position, with the dorsal, ventral, cephalad and caudad directions. FIG. 2 shows one of a vertebra of this spinal portion viewed along the longitudinal axis, looking in the caudal direction. Features that are visible in these views include the vertebral body 10 and the spinal canal, defined by the vertebral foramen 12 and the intervertebral foramen 14, in which the spinal column (not shown) is positioned. A pair of intervertebral discs 16 is shown, as well as the pedicle 18, the transverse processes 20, the superior articular process 22, the inferior articular facet 24 and a superior articular facet 26. Also visible is a lamina 28 and the spinous process 30. Positioned between adjacent spinous processes 30 are the interspinous ligament 32 and the supraspinous ligament 34.

The interspinous ligament 32 is a midline structure contiguous with adjacent spinous processes 30. The interspinous ligament 32 moves by virtue of elongation and delongation during spinal flexion and extension. This elastic movement of the ligament 32 provides for cephalo-caudal movement of lesser magnitude at any instantaneous point in the ligament during said flexion and extension. By their bony nature, the spinous processes 30 move relatively non-elastically, and thereby change position to a greater magnitude during flexion and extension.

In the following embodiments, a spinal implant is tethered to the posterior mammalian, and preferably, human spine of the type shown in FIGS. 1 and 2. The tethering is achieved by a tethering device that is placed between two adjacent lumbar vertebral spinous processes 30, through the interspinous ligament 32. The tethering device allows secure placement of a spinal implant, such as a dorsal column stimulator (DCS) or a medication pump (MP). Of course, the tethering devices to be described herein are not limited to only these spinal implants, or placement at one level or only at the lumbar level. Cervical and thoracic placement, as well as use for tethering of other spinal implants can be easily accommodated by this tethering device and method as a natural progression of this device and method. The spinal implants discussed herein are those known in the prior art and those that may be developed, and this application is not directed at the implants themselves. The application is directed at the means for tethering the implants.

In the surgical placement of spinal implant such as a dorsal column stimulator or a medication pump, a conduit must be subcutaneously tunneled from a remote unit to the spinal implant. In the case of a dorsal column stimulator, the conduit is typically an insulated wire for delivering electrical current from a battery. In the case of a medication pump, the conduit is typically a catheter that delivers a liquid medication from a reservoir. Tethering the conduit to a fixed point maintains the spinal implant in proper position in or about the spinal canal which maintains proper function of the spinal implant. It will be recognized that not only the spinal implant will need to be tethered, but the conduit should also be tethered along the spine from its source to the spinal implant.

The ability of the elastic interspinous ligaments to reduce the magnitude of the cephalo-caudal migration due to motion, as described above, makes the interspinous ligaments the preferred site for tethering over attachment to the spinous process or other posterior bony attachment, provided that a secure attachment can be made there.

A tethering device as described in the embodiments herein, especially one that uses quick connection mechanisms, eliminates the need for suturing and reduces the incision size, thereby reducing the operative time and healing time. Some quick connection mechanisms allow the connection to “float”. These quick connect mechanisms allow adaptability towards a fair lead for the spinal implant, reducing the repetitive stress on the tethered portion of the spinal implant during flexion extension motion. The focus of the invention as described herein lies not in specific quick connect mechanisms as much as the concept of using quick connect mechanisms to tether a spinal implant to the posterior spine structures is.

### The Tethering Device Components

A tether device according to one of the embodiments should comprise both a horizontal element and an implant connector, which connects to the spinal implant or conduit. The horizontal element will be referred to in some instances herein as the “first element” with the implant connector being referred to occasionally as the “second element.”

The horizontal element, in the embodiments described herein, has one or more beams or rods that are adapted for placement that is horizontal to the long axis of the spine, or approximates such horizontal placement.

In some instances, the implant connector is integrally formed with horizontal element. In some other instances, the implant connector is rigidly attached to the horizontal element, and, in yet further instances, the implant connector is allowed to float in semi-rigid relationship with the horizontal element. The particular choice among these options will depend upon surgeon preference and the use of a particular quick connect mechanism.

In many of the disclosed embodiments, the horizontal element will cross the spinal midline through the interspinous ligament. It may also pass through other posterior spinal structures, such as bone, including the spinous process.

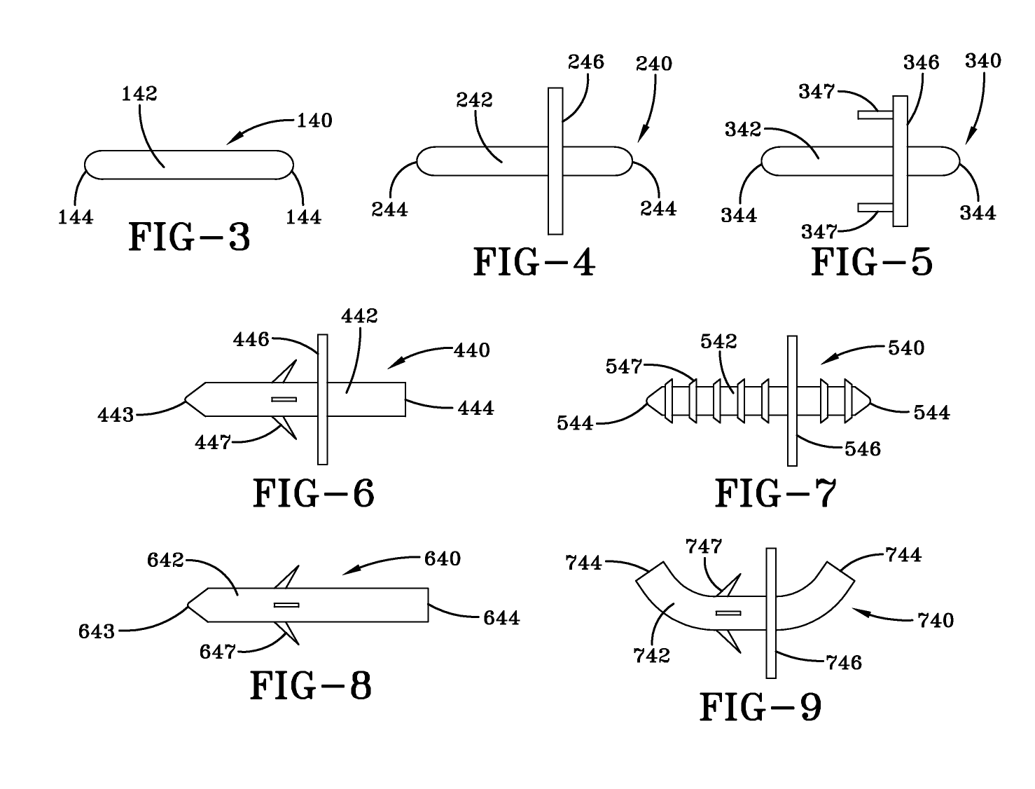

Various embodiments for the first, or horizontal, element are disclosed in side elevation view in FIGS. 3 through 9, which will now be described in more detail.

A first embodiment 140 of a first element is seen in FIG. 3. In this embodiment, the first element 140 is an elongate cylindrical body 142 with a generally smooth exterior surface. The most notable feature is a pair of generally rounded ends 144.

A second embodiment 240 of a first element is seen in FIG. 4. In this embodiment, the first element 240 is an elongate cylindrical body 242 with a generally smooth exterior surface. In addition to a pair of generally rounded ends 244, the second embodiment 240 has a radial flange 246 fixed along the length of the body 242. In the particular example, the flange 246 is not centered along the length of the body 242. Instead, it is shifted somewhat from the center. Flange 246 generally serves as a “stop” to delimit penetration of the body 242 through the body part, typically the interspinous ligament.

A third embodiment 340 of a first element is seen in FIG. 5. In this embodiment, the first element 340 is an elongate cylindrical body 342 with a generally smooth exterior surface. In addition to a pair of generally rounded ends 344, the second embodiment 340 has a radial flange 346 fixed along the length of the body 342. In the particular example, the flange 346 is not centered along the length of the body 342, but is, instead, shifted somewhat from the center. Flange 346 generally serves as a “stop” to delimit penetration of the body 342 through the body part, typically the interspinous ligament. Further, embodiment 340 is distinguished from embodiment 240 by the added penetration elements 347 that extend normally from one face of the flange 346 and provide additional stability to the tethering connection established. Although two elements 347 are shown, there could be only one such element or there could be more, depending upon the particular design.

A fourth embodiment 440 of a first element is seen in FIG. 6. In this embodiment, the first element 440 is an elongate cylindrical body 442 with a generally smooth exterior surface. Instead of a pair of generally rounded ends, a first end 443, intended for penetration, is somewhat more pointed, while a second end 444 is blunt. In addition to a radial flange 446 fixed along the length of the body 442, the embodiment 440 also has a plurality of angled barbs 447 formed along the body. These barbs 447 are intended to delimit movement of the embodiment 440 bi-directionally, once the embodiment is penetratingly positioned through the body part.

A fifth embodiment 540 of a first element is seen in FIG. 7. In this embodiment, the first element 540 is an elongate cylindrical body 542. Both ends 544 of the body 542 are somewhat pointed, although with a rounded rather than pointed tip, indicating their capacity for penetrating body tissue. In addition to a radial flange 546 fixed along the length of the body 542, the embodiment 540 also has a plurality of frustoconical retention elements 547 formed along the body. These elements 547 are oppositely directed on the two sides of the radial flange 546, with the larger end of each frustoconical element facing toward the radial flange, so that they can provide bi-directional delimitation of movement, acting in concert with the radial flange, once the embodiment 540 is penetratingly positioned through the body part.

A sixth embodiment 640 of a first element is seen in FIG. 8. In this embodiment, the first element 640 is an elongate cylindrical body 642 with a generally smooth exterior surface. Instead of a pair of generally rounded ends, a first end 643, intended for penetration, is somewhat more pointed, while a second end 644 is blunt. Unlike fourth embodiment 440 of FIG. 6, this embodiment 640 lacks the radial flange 446, but has a plurality of angled barbs 647 spaced angularly around the body 642 at a selected radial location. These barbs 647 are intended to delimit movement of the embodiment 640 once the embodiment is penetratingly positioned through the body part.

Another variation of the fourth embodiment 40 of a first element is seen as seventh embodiment 740 in FIG. 9. In this embodiment, the first element 740 is an arcuate cylindrical body 742 with a generally smooth exterior surface. It has a pair of generally blunt ends 744. Like embodiment 440 of FIG. 6, this embodiment 740 possesses a radial flange 746, as well as a plurality of angled barbs 747 spaced angularly around the body 742 at a selected radial location. These barbs 747 are intended to delimit movement of the embodiment 740 bi-directionally, once the embodiment is penetratingly positioned through the body part.

Each first or horizontal element 140, 240, 340, 440, 540, 640, 740 may be provided with at least one quick-attachment means for either attaching an implant connector or for attaching a spinal implant directly. In some cases, the quick-attachment means will be provided on the implant connector or the spinal implant.

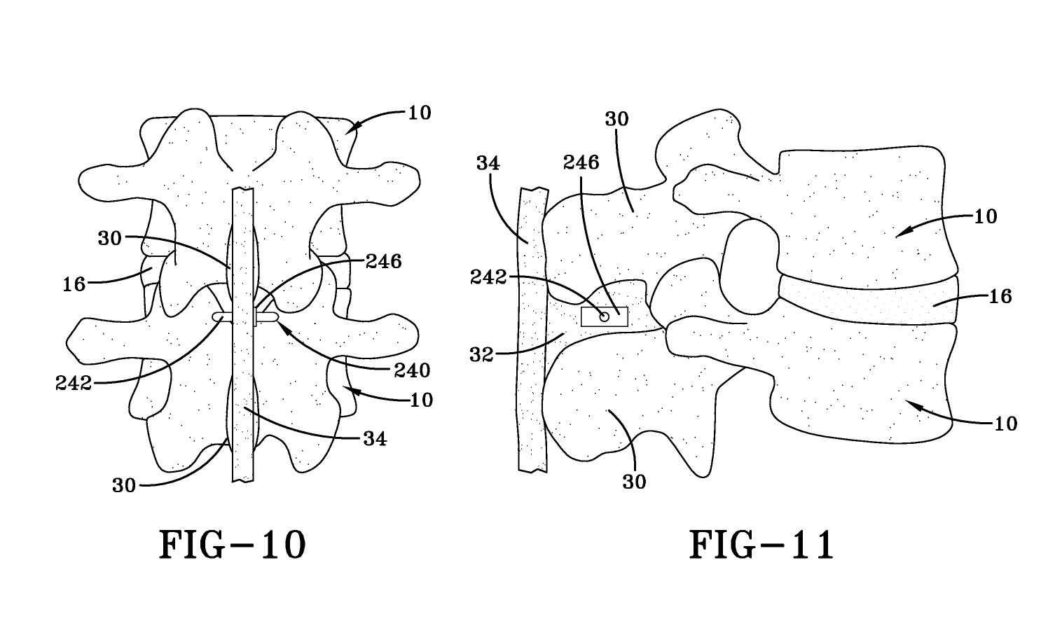

FIGS. 10 and 11 depict the attachment of a first or horizontal element 240 into a spine, particularly at the interspinous ligament. FIG. 10 is a view looking onto the dorsal surface with spinal structures such as the spinous process 30, the vertebral body 10, the supraspinous ligament 34 and a vertebral disc 16 visible. FIG. 11 is a side view of the same implantation, as viewed from the right side of FIG. 10, further showing the radial flange 246 and body 242 of the first element 240, as well as the interspinous ligament 32, which is not seen in FIG. 10.

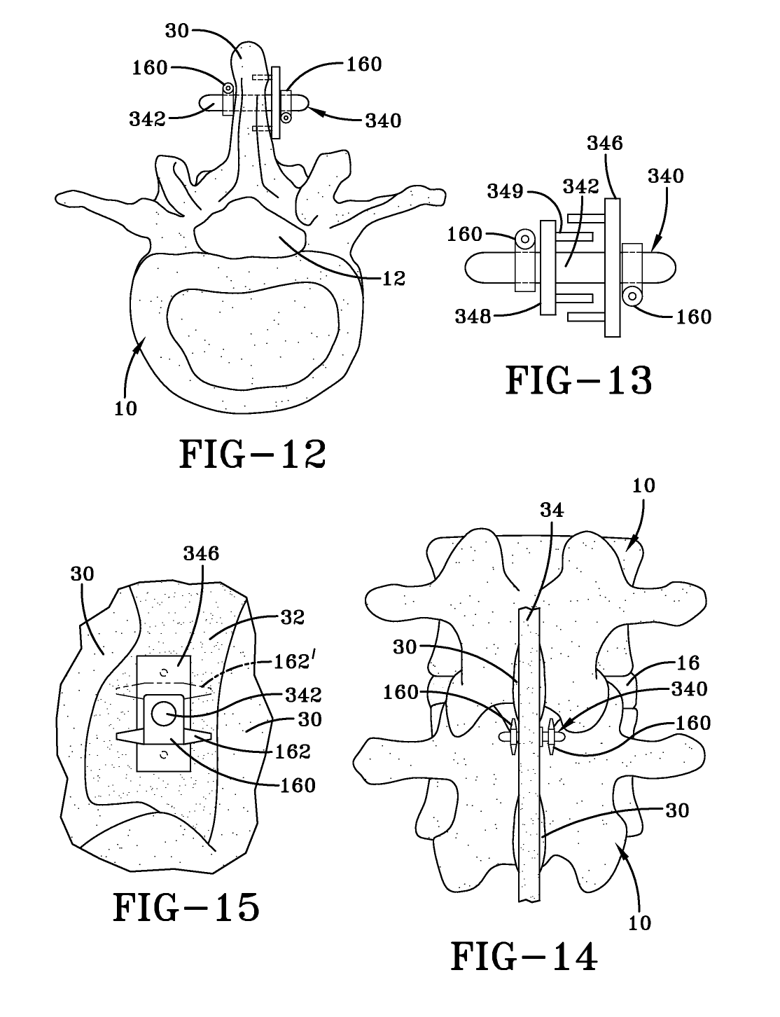

Once a first element, such as 340 is implanted into the spine, as is illustrated in FIG. 12 in a view along the longitudinal axis of the spine, the exposed portions of the body 342 may be populated with implant connectors, such as the implant connector embodiment 160 shown. Spinal structures depicted include the vertebral foramen 12, the spinous process 30 and vertebral body 10, which assist in orienting the viewer. In the depiction of FIG. 12, the first element 340 has a pair of implant connectors 160 attached to it along body 342. In this depiction, the quick connection means is provided on the implant connector 160 and serves to fix the implant connector to the first element 340.

In attaching an implant connector, for example, connector 160, to a first element such as 340, the attachment may be made rigidly, semi-rigidly or non-rigidly. In a rigid attachment, the spinal implant is constrained from movement relative to the first element 340. In a non-rigid attachment, the spinal implant has at least limited angular rotation available around the axis of the first element's elongate body, which allows adaptability towards a fair lead of the spinal implant during spinal bending. In a semi-rigid attachment, the spinal implant can rotate about an axis essentially parallel to the spinal axis. The determination of whether an attachment should be rigid, semi-rigid, or non-rigid device is a matter of the surgeon's professional judgment.

FIG. 13 shows, isolated from the spine structures, an alternate assembly of the tethering device in which a second radial flange 348, having penetration elements 349, is positioned onto the body 342 on the side of the interspinous ligament opposite the radial flange 346. As previously, implant connectors 160 are shown in place on the body 342.

FIG. 14, which may be compared to readily to FIG. 10, shows, in a plan view of the dorsal surface, the tethering device after a pair of implant connectors 160 are attached to the first element 340. As in FIG. 10, spinal structures such as the spinous process 30, the vertebral body 10, the supraspinous ligament 34 and a vertebral disc 16 are visible.

FIG. 14 shows further features of implant connector 160 and depicts how this connector, in the non-rigid attachment described above, is able to rotate about the axis of the body 342. Radial flange 346 and interspinous ligament 32 are shown to provide orientation. The implant connector 160 has an implant-receiving portion 162 that can rotate from the position shown to the position shown as 162′.

Implant connectors such as 160 may be placed on the first element, such as 340, after placement of the first element into the spine, and, depending upon the exact geometry of the first element 340. For example, placement of the implant connector 160 on the left side of FIG. 12 on the first element prior to placement of the first element on the spine is very unlikely, but placement of the implant connector on the right side of FIG. 12 is much more likely.

The attachment of the spinal implant and/or a conduit associated with a spinal implant can occur through a variety of means. These would include chemical bonding, particularly with a bio-acceptable adhesive such as a cyanoacrylate or a silicone, or mechanical means, particularly compressive means, as well as combinations thereof. Accordingly, the quick connect means for attaching the implant connector to the first element is also variable, depending upon the level of the rigidity desired.

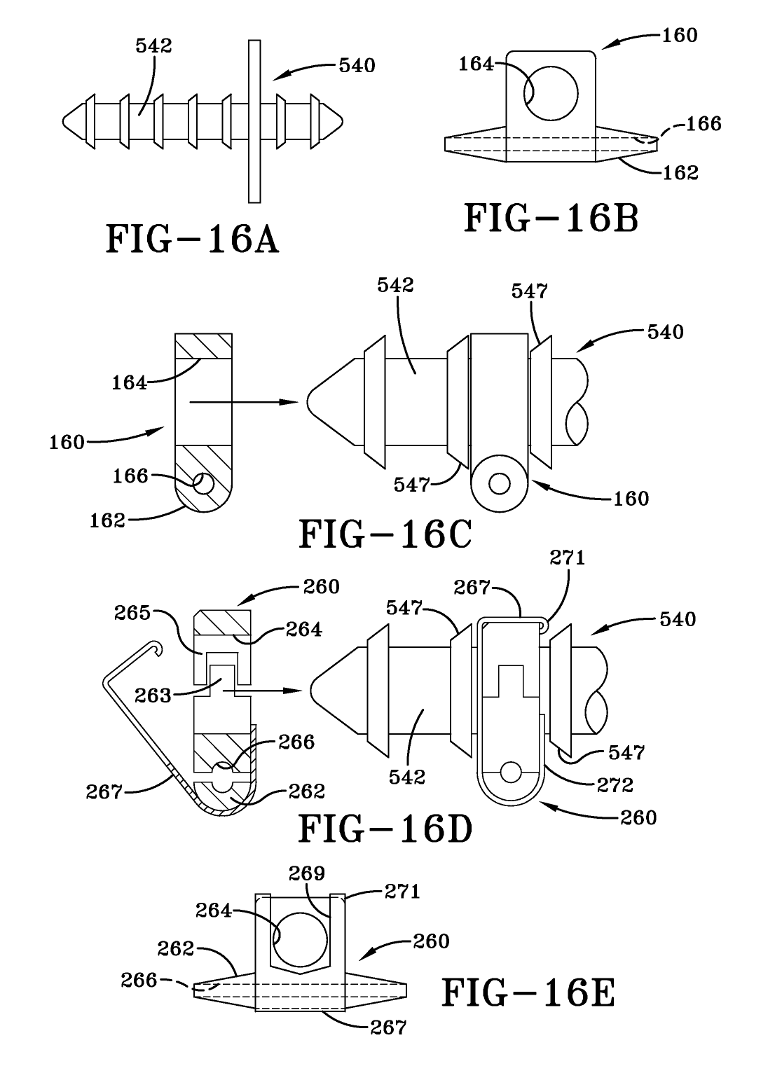

FIGS. 16A through 16E illustrate a few variations of how the attachment of an implant connector to a first element may be made. FIG. 16A shows a first element 540 as seen in FIG. 7. FIG. 16B shows a front sectional view of an implant connector 160, as seen previously in FIG. 12. Notable in FIG. 16B are a through hole 164, sized and adapted to be received on body 542 of the first element 540 and implant-receiving portion 162, with a through hole 166 in which a conduit may be received shown in side section. Through holes 164 and 166 are in non-intersecting orthogonal relationship to each other. FIG. 16C shows how the implant connector 160 is slidingly fitted onto body 542, with frustoconical retention elements 547 deforming sufficiently to permit the implant connector 160 to be received in a journalled relationship between a pair of adjacent retention elements 547. It will be clear that spaced-apart barbs, as seen in FIGS. 6, 8 and 9, could also provide an equivalent retention of the implant connector 160. FIG. 16D shows a variation on this attachment, using an implant connector 260. Implant connector 260 is similar to implant connector 160 of FIG. 16B, but it has important differences. Through hole 264 does not have a fixed diameter. It has a diameter that varies through the interaction of elements 263 and 265. Also, through hole 266 also has a variable diameter, due to the longitudinal split along implant-receiving portion 262. Also, implant connector 260 has a clip 267 that has been fenestrated with cutout 269, both of which are best seen in FIG. 16E, which shows the implant connector of FIG. 16D, viewed from the left side of that figure. Clip 267, as seen in the right side of FIG. 16, has portions 271, 272 that wrap around the body of implant connector 260, compressing the attachment of at least one of: through hole 264 to body 542 and through hole 266 to a spinal implant (not shown in FIG. 16D) placed therethrough.

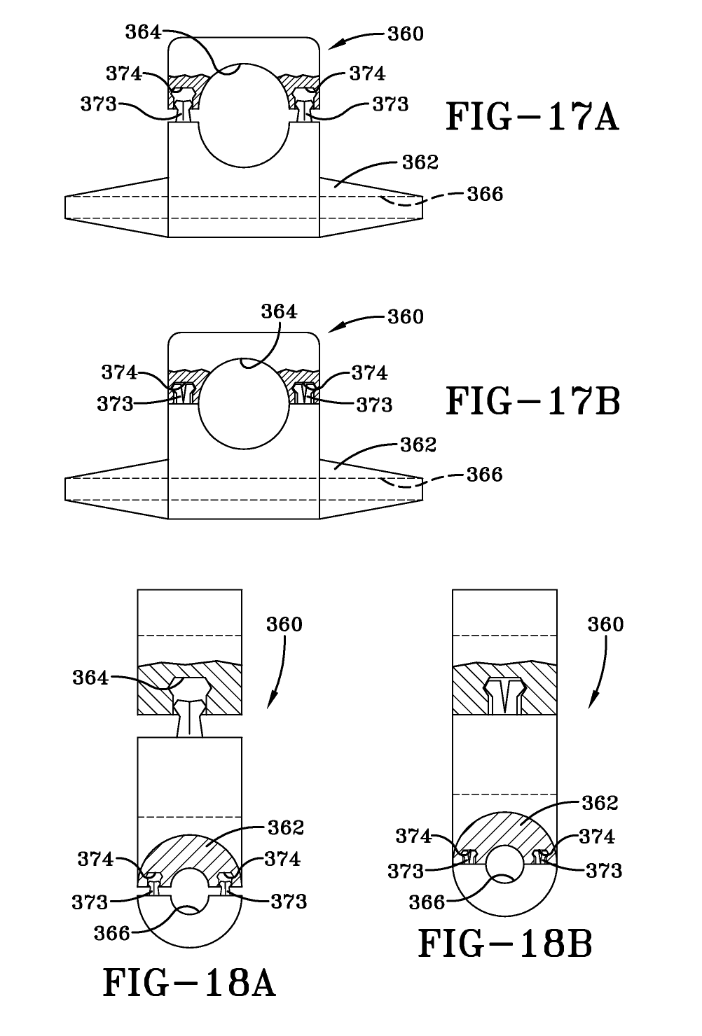

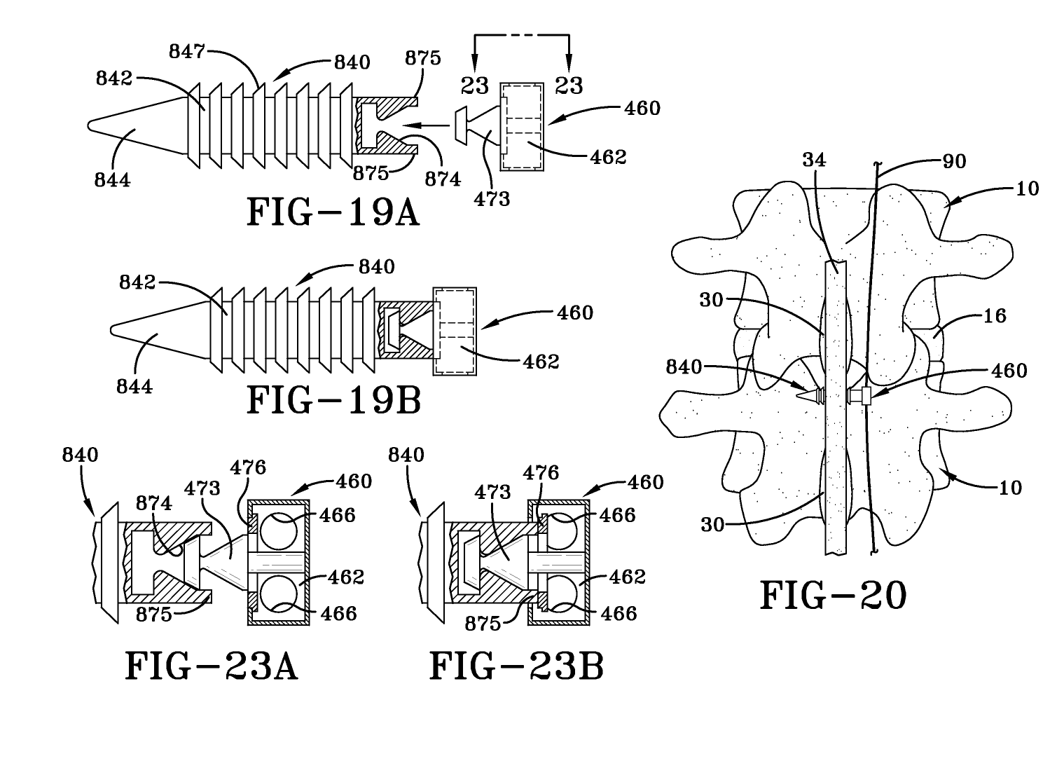

FIGS. 17A, 17B, 18A and 18B show front and side elevation views of a third embodiment 360 of an implant connector. Many structures are common with the prior embodiments and will be readily recognized, such as implant-receiving portion 362 and through holes 364 and 366. Deformable pins 373 and corresponding openings 374 allow each of the through holes 364, 366 to be changed from the “open” position of FIGS. 17A, 18A to the “closed” position of FIGS. 17B, 18B, respectively.

FIGS. 19A and 19B show a further embodiment 840 of a horizontal element and a further embodiment 460 of an implant connector, with FIG. 19A showing the parts in an unassembled state and with FIG. 19B showing the parts assembled in a manner that not only secures the implant connector to the horizontal element, but which also places compressive force upon one or more through holes (not shown in FIG. 19) in the implant connector. Horizontal element 840 has a body 842 that is adapted at a first end 844 for penetrating attachment to an interspinous ligament. Horizontal element 840 is adapted at a second end with one element 874 of a mating set of quick connect means, particularly, a shaped opening for receiving a deformable pin 473 that is formed on the implant connector 460 and operates as a second element of the mating set. Deformable pin 473 is directly attached to an implant-receiving portion 462 of the implant connector 460. Body 842 is provided with frustoconical retention elements 847. The second end of body 842 also has extending element or elements 875 that are used in the placement of compressive force on the through holes in the implant connector, as explained in more detail below.

FIG. 20 is a plan view of the dorsal surface of a spine, depicting deployment of a tethering device comprising horizontal element 840 and implant connector 460 through the interspinous ligament. Spinal structures depicted include vertebral bodies 10 and their associated spinous processes 30, a vertebral disc 16 and the supraspinous ligament 34 that overlies the interspinous ligament. Also shown in FIG. 20 is a conduit 90 that is part of the spinal implant, the conduit being compressively retained in the implant connector.

FIGS. 21A and 21B show a further embodiment 940 of a horizontal element and a further embodiment 560 of an implant connector, with FIG. 21A showing the parts in an unassembled state and with FIG. 21B showing the parts assembled in a manner that not only secures the implant connector to the horizontal element, but which also places compressive force upon one or more through holes (not shown in FIG. 21) in the implant connector. Horizontal element 940 is a bone screw with a body 942 that is threaded for penetrating attachment into a spinous process. Horizontal element 940 is adapted at its head end with one element 974 of a mating set of quick connect means, particularly, a shaped opening for receiving a deformable pin 573 that is formed on the implant connector 560 and operates as a second element of the mating set. As shown in FIG. 21A, it would be typical to also provide the head end of the bone screw with a drive fitting 975 for receiving a corresponding drive element for rotatingly driving the bone screw into the spinous process (or other bony material). Deformable pin 573 is directly attached to an implant-receiving portion 562 of the implant connector 560. The head end of body 942 also has extending element or elements 975 that are used in the placement of compressive force on the through holes in the implant connector, as explained in more detail below.

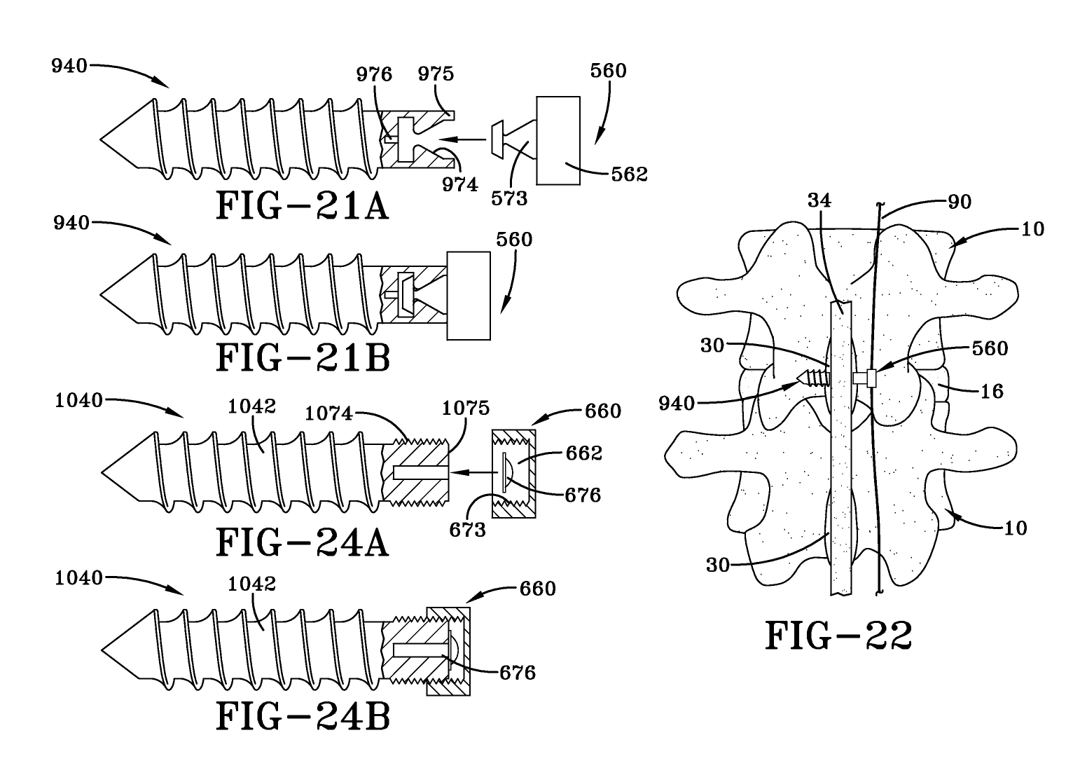

FIG. 22 is a plan view of the dorsal surface of a spine, depicting deployment of a tethering device comprising horizontal element 940 and implant connector 560 into the spinous process. Spinal structures depicted include vertebral bodies 10 and their associated spinous processes 30, a vertebral disc 16 and the supraspinous ligament 34 that overlies the interspinous ligament. Also shown in FIG. 22 is a conduit 90 that is part of the spinal implant, the conduit being compressively retained in the implant connector.

FIGS. 23A and 23B are enlarged end sectional elevation views of a portion of FIGS. 19A and 19B, respectively, to show how the engagement of the mating elements 473, 874 serve to place compressive force on one of more through holes 466 in implant connector 460. The notable feature is how extending element or elements 875 on horizontal element 840 engage an element 476 seated in a slot of implant-receiving portion 462 and move the element into a position (as seen in FIG. 23B) where it obstructs a portion of the otherwise open diameter of the one or more through holes 466. The same type of means would be used for applying compressive force to a conduit passing through the through holes of the embodiment 560 shown in FIGS. 21A and 21B, so an express depiction of that means is not presented, as an understanding of FIGS. 23A and 23B suffices.

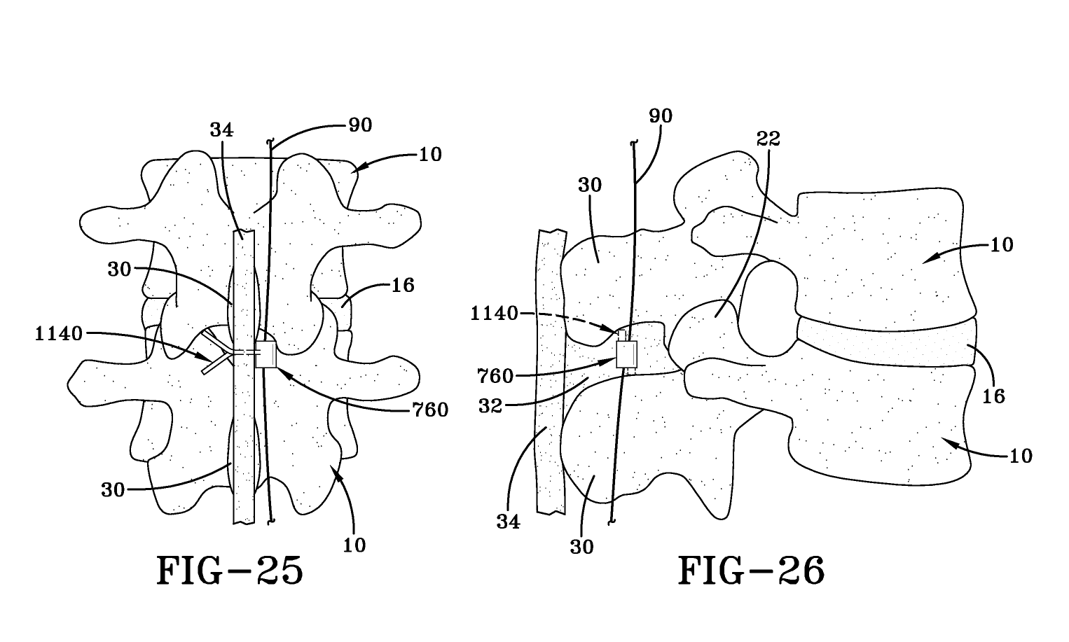

FIGS. 24A and 24B show a further embodiment 1040 of a horizontal element and a further embodiment 660 of an implant connector, with FIG. 24A showing the parts in an unassembled state and with FIG. 24B showing the parts assembled in a manner that not only secures the implant connector to the horizontal element, but which also places compressive force upon one or more through holes (not shown in FIG. 24) in the implant connector. In the embodiment shown, horizontal element 1040 is a bone screw with a body 1042 that is threaded for penetrating attachment into a spinous process. Horizontal element 1040 is adapted at its head end with one element 1074 of a mating set of quick connect means, particularly, male threading to be received in female threading 673 that is formed inside the implant connector 660 and operates as a second element of the mating set. The head end of body 1042 also has extending element or elements 1075 that engage an element 576 seated in implant-receiving portion 662 and move the element into a position (as seen in FIG. 23B) where it obstructs a passage through the implant-receiving portion that receives a conduit of a spinal implant (not shown).

FIGS. 25 and 26 depict the attachment of a first or horizontal element 1140 with an attached implant connector 760 into a spine, particularly at the interspinous ligament.

FIG. 25 is a view looking onto the dorsal surface with spinal structures such as the spinous process 30, the vertebral body 10, the supraspinous ligament 34 and a vertebral disc 16 visible. Conduit 90 that is captured in the implant connector 760 is also seen. FIG. 26 is a side view of the same implantation, as viewed from the right side of FIG. 10. In this embodiment, a large portion of the body 1142, particularly extending from the end opposite implant connector 760, is divided longitudinally, so that the resulting legs may be malleably separated after the body has penetrated the interspinous ligament 32, thereby retaining the element 1140 in the ligament. In this case, implant connector 760 may be integrally formed on horizontal element 1140 or may be connected by one of the various quick connect means that have been taught herein.

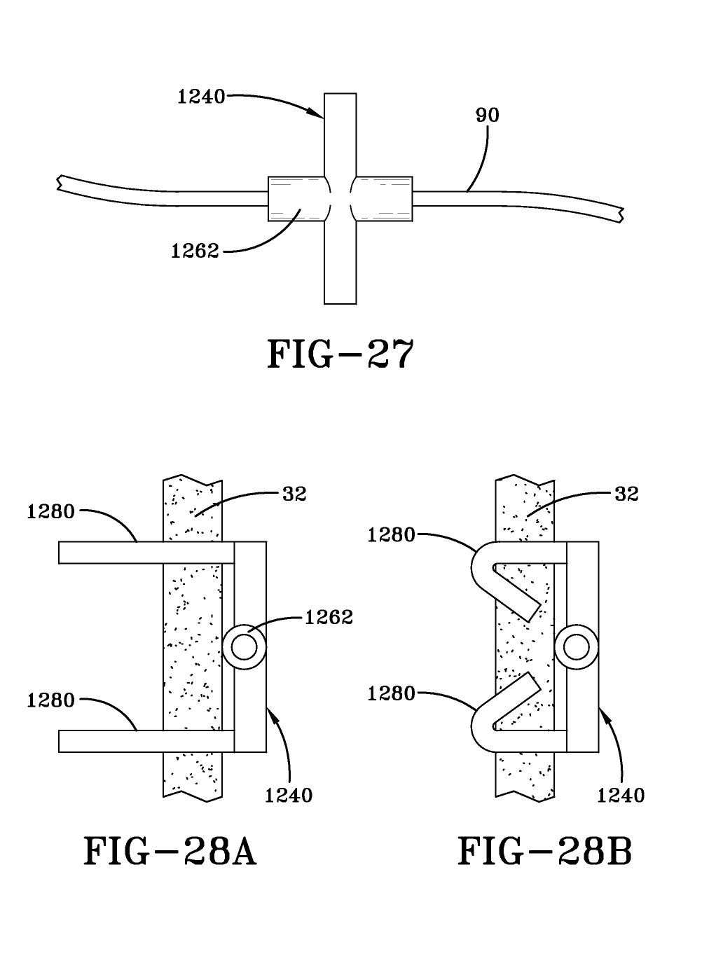

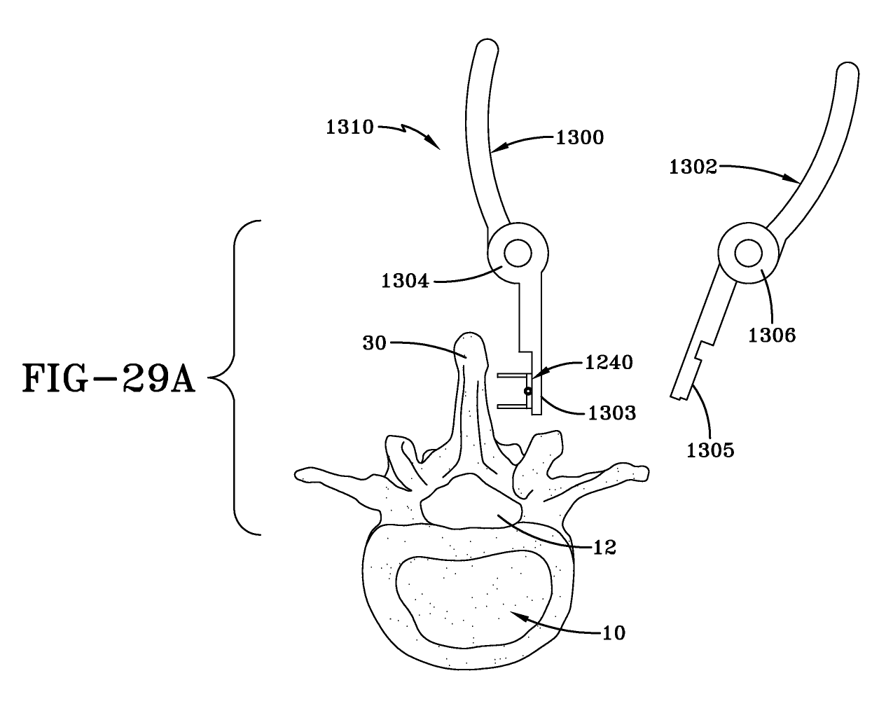

In another application of the inventive method, a staple like tethering device 1240 incorporates a horizontal element with an integral implant connector, the latter having an implant-receiving portion 1262 in the nature of a tunnel with a collapsible portion. A particular use is found with regard to a dorsal column stimulator implant (DCS) as discussed above for tethering the DCS power supply lead wire to the dorsal spine. After placing the DCS wire electrode at the correct position of application of stimulation current (a technique well known to those practiced in the art), the DCS wire is passed through the tunnel 1262 of the tethering device 1240. The device 1240 is then passed down to the interspinous ligament 32 in the preferred position, through a dorsal incision. FIG. 27 shows a front elevation view of the device 1240, and FIGS. 28A and 28B show, in side elevation view, how the device is passed through the interspinous ligament and then secured thereto. Particularly, the device 1240 has a pair of staple-like barbs 1280, which pass in a straightened condition through the ligament (FIG. 28A) and are then deflected towards each other (FIG. 28B). FIG. 27 also shows the conduit 90.

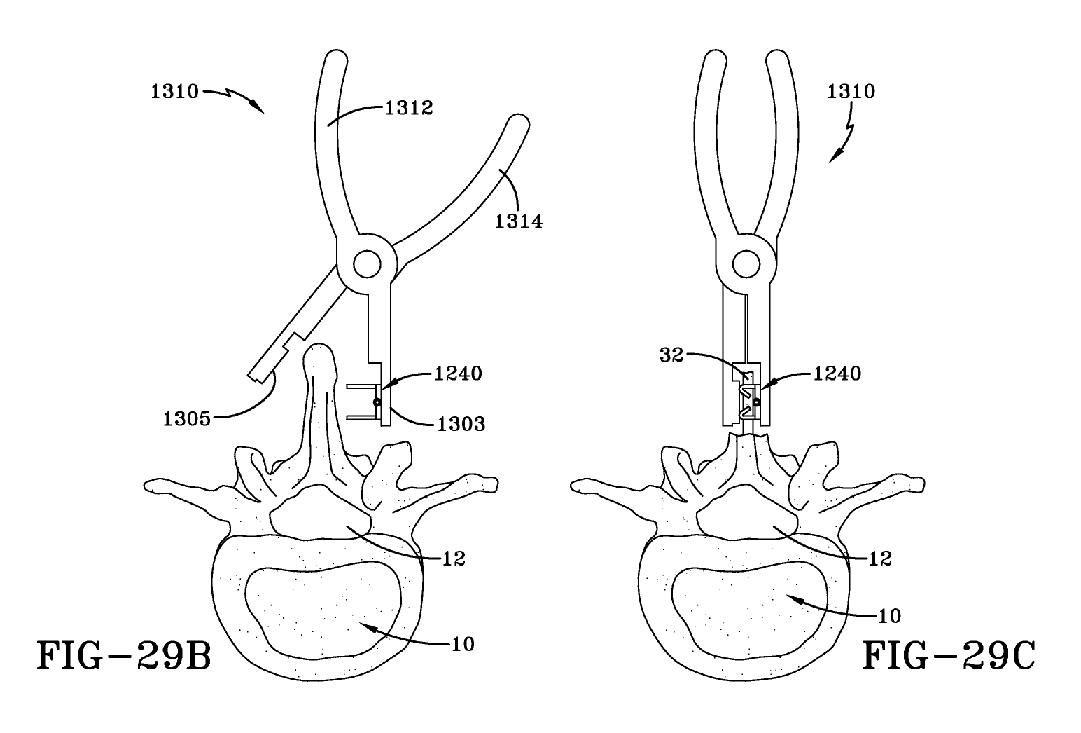

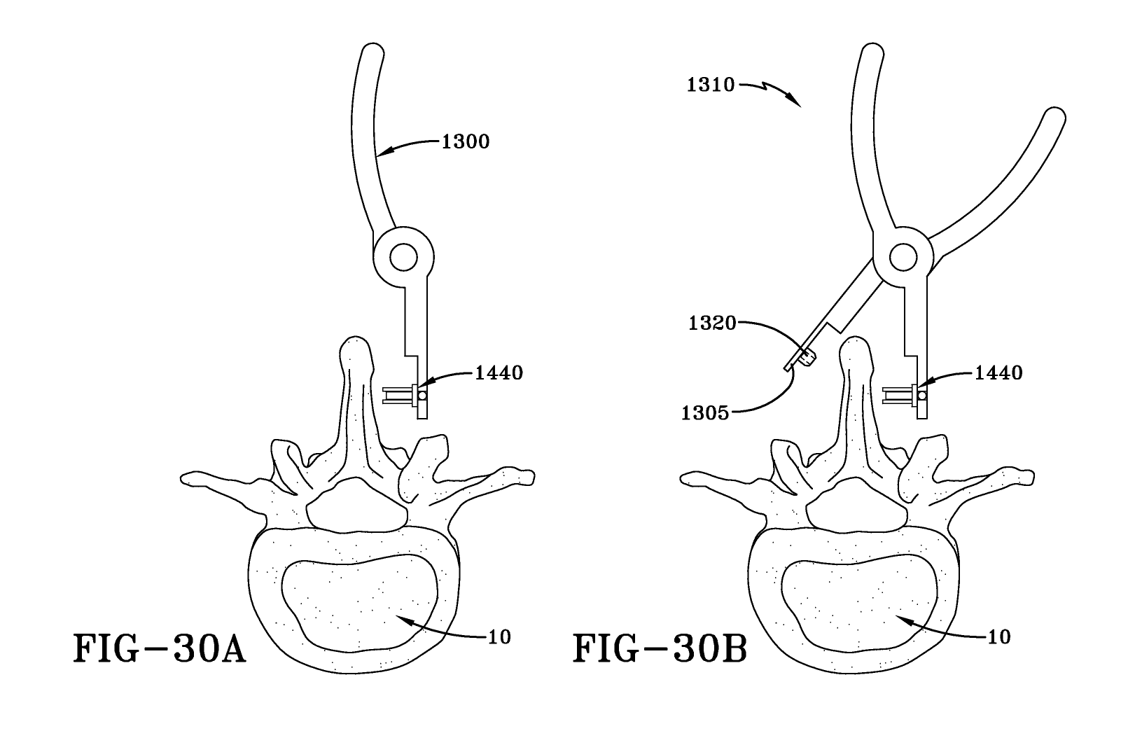

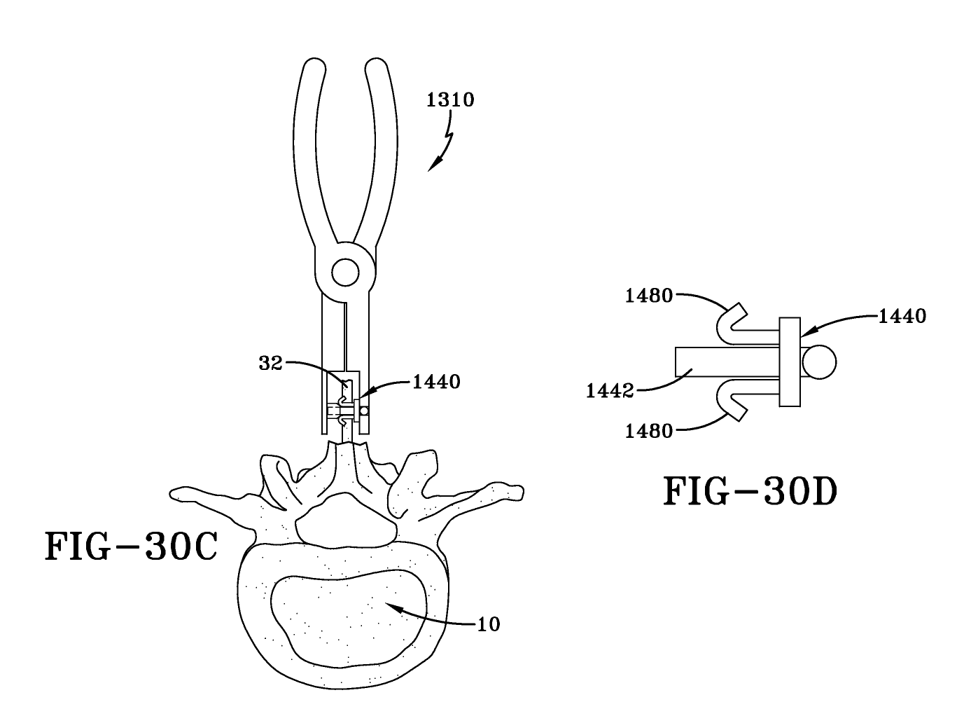

Further understanding of the placement technique will be understood through reference to FIGS. 29A, 29B and 29C. As seen in a view taken down the longitudinal axis of the spine, the tethering device 1240 may be retainingly seated by the first piece 1300 of a two piece stapling gun 1310. The first piece 1300, at a lower portion 1303 thereof, holds the device 1240 (with the DCS wire in the tunnel) for passage into the dorsal incision and then through the interspinous ligament (not shown, but the site of which is identifiable from landmarks such as the spinous process 30, the vertebral foramen 12 and the vertebral body 10). As illustrated, the tethering device 1240 will pass through the interspinous ligament in a right to left direction. Once the tethering device 1240 is engaged through the interspinous ligament, a second piece 1302 of the stapling gun 1310 is passed into the left side of the interspinous ligament and the stapling gun is assembled by attaching corresponding pivot portions 1304, 1306 on the respective stapling gun pieces 1300, 1302. This attachment of the two pieces 1300, 1302 results in the configuration shown in the same aspect view in FIG. 29B. A lower portion 1305 of the second piece 1302 helps to align the second piece with the protruding barbs 1280 of the tethering device 1240. By compressing the upper portions 1312, 1314 together, the lower portions 1303, 1305 are compressed, folding the staple barbs 1280 towards each other, in a fashion well know to anyone who has knowledge of a standard desktop stapler. As this occurs, the implant receiving portion 1262 is compressed against the interspinous ligament, and the collapsible portion collapses, capturing the conduit 90. A slow curing bonding agent maybe added to the implant-receiving portion 1262 to assist in retaining the conduit 90. Additionally, although not preferred, the wire may be held in the tunnel through the use of a suture/ligature.

A variation of the placement technique is also shown in FIGS. 30A, 30B, 30C and 30D. In drawings that are analogous to FIGS. 29A through C, a rivet-like embodiment 1440 of the tethering device is shown. A central rod 1442 of the tethering device is received in a tube 1320 of the lower portion 1305, in a manner so that barbs 1480 are turned away from each other rather than towards each other. FIG. 30D shows the configuration of the tethering device 1440 after placement.

What is claimed is:

1. A device for tethering an implant device along the posterior spine of a mammal, particularly a human, comprising:
a first element, adapted to be secured to an interspinous ligament of the mammal;
a second element, adapted for attachment to the implant device; and
means for fixing the first element to the second element in a quick-connect manner.

2. The device of claim 1, wherein:
the first element comprises a beam adapted for placement through the interspinous ligament in a direction substantially horizontal to a long axis of the spine.
